(19) United States
(12) Patent Application Publication
(10) Pub. No.: US 2002/0173739 A1
(43) Pub. Date: Nov. 21, 2002

(54) Footwear brace

(76) Inventor: Clint Stanley Scott Jensen, Woodridge (AU)

Correspondence Address: Frederick H Colen, Reed Smith, PO Box 488, Pittsburgh, PA, 15230, US

(21) Appl. No.: 10/070,703

(22) PCT Filed: Jul. 11, 2001
(86) PCT No.: PCT/AU01/00832

(30) Foreign Application Priority Data
Jul. 11, 2000 (AU) PQ 8720

(51) Int. Cl. A61F 5/00

(52) U.S. Cl. 602/27

(57) ABSTRACT

The invention concerns an article used in conjunction with footwear. Specifically, the invention provides a footwear brace (1) comprising a heel strap (2), a length adjustable sole strap (3), and a length adjustable and tensionable instep strap (4). Braces are typically provided as pairs comprising left and right braces.

### TECHNICAL FIELD

This invention relates to footwear, particularly footwear used in conjunction with sporting activities such as skating. More particularly, the invention relates to a brace for such footwear.

### BACKGROUND ART

A common problem with footwear such as the boot of an ice or roller skate is that with prolonged use the boot can become loose on the user's foot. This can have an adverse effect on the skater's performance and can even result in injury through inadequate control of the skate. Because of the cost of the boot (if detachable) or the skate as a whole (if the boot is integral with the skate), replacement is not always an option. Consequently, strapping is applied to give better support to the foot.

The strapping mentioned in the previous paragraph is applied either by securing it to the boot or by affixing it with an adhesive. This does not always afford the required degree of bracing and in time the strapping often losses its effectiveness. Furthermore, strapping can be unsightly.

It would therefore be desirable to have a brace for footwear such as the boot of a skate which is easy to fit and which gives the required degree of bracing to the foot of a user.

### SUMMARY OF THE INVENTION

The object of the invention is to provide a brace that meets the requirements set out in the previous paragraph and which overcomes the disadvantages of known methods of bracing footwear.

In a broad format the invention provides a footwear brace comprising a heel strap, a length adjustable sole strap, and a length adjustable and tensionable instep strap, wherein one end of each of said heel, sole and instep straps are joined at a common point and the other end of each of said heel, sole and instep straps are joined at another common point.

It will be appreciated from the preceding paragraph that a brace according to the invention comprises an article with integral heel, sole and instep members that strap the piece of footwear to the foot of the wearer in the ankle region. Because the brace comprises three straps with ends that are joined at opposite sides of the foot, triangulated bracing is afforded in both longitudinal and lateral planes.

The straps of a brace according to the invention can be fabricated from any suitable flexible material. Typically, straps comprise a thermosetting plastics material. The straps in this case are usually formed by injection moulding.

The adjustability and tensionability of the straps of the brace can be by any of the methods known to those of skill in the art of preparing straps for footwear and the like. Advantageously, the adjustability is by virtue of the relevant strap comprising two portions that can be releasably fixed to each other. This aids fitting of the brace to footwear as will be explained in greater detail below.

The releasable fixing typically comprises a plurality of pins on one strap portion that can be received by complementary holes in the other (overlapping) strap portion. Another form of releasable fixing is patches of hook and loop material on the strap portions. Such material is commercially available as VELCRO™

Adjustability of a strap can also be effected by providing a clasp or buckle through which an end of the strap is passed. Such a manner of adjustment of the strap also allows tensioning of the strap.

In a preferred form of brace, ends of the heel strap and sole strap are pivotally joined. This allows greater adaptability to the footwear and foot of the wearer to which the brace is fitted.

Because of the adjustability of straps of a brace according to the invention, the brace can be used with a range of footwear sizes. However, braces will normally be sized appropriately for use with children's footwear, or small, medium or large adult footwear. Braces are also advantageously provided in pairs, there being left and right handed forms.

Having broadly described the invention, a brace will now be exemplified with reference to the drawings briefly described hereafter.

### BRIEF DESCRIPTION OF THE DRAWINGS

FIG. 1 is a perspective view of a brace designed for the right foot of a user.

FIG. 2 is a perspective view of the complementary (left-hand) brace to the FIG. 1 brace with adjustable straps disengaged.

FIG. 3 is a perspective view of the brace of FIG. 1 in position on the foot of a wearer.

In the FIG , the same item number is used for a feature included in more than one drawing. A particular drawing is not necessarily to the same scale as other drawings.

### BEST MODE AND OTHER MODES OF CARRYING OUT THE INVENTION

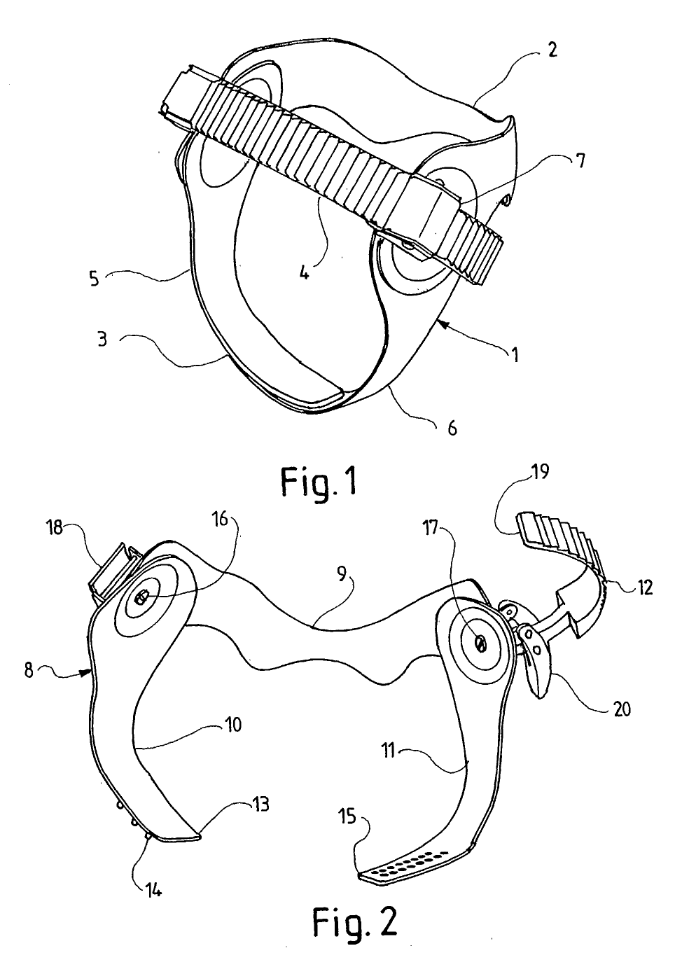

With reference to FIG. 1, there is shown right-handed brace 1 comprising heel strap 2, sole strap 3 and instep strap 4. Sole strap 3 consists of two overlapping portions, 5 and 6, the function of which will be explained below. Instep strap 4 has a serrated outer surface which can be fixed in a desired position by releasable clip 7 thereby making the strap length adjustable.

The complementary, right-handed, brace to that shown in FIG. 1 is presented in FIG. 2. However, in the latter figure the brace is effectively “opened up” so components can be more clearly seen. FIG. 2 shows brace 8 comprising heel strap 9, portions 10 and 11 of a sole strap, and instep strap 12. End 13 of sole strap portion 10 includes a plurality of outwardly extending pins one of which is indicated at 14, that are received in a selected number of corresponding holes in end 15 of sole strap portion 11. In this way, the sole strap can be broken for fitting the brace yet is length adjustable by virtue of there being a much greater number of holes in end 15 than there are pins on end 13 of the other sole strap portion. Sole strap portions 10 and 11 are pivotally joined to opposite ends of heel strap 9 via rivets 16 and 17, respectively.

With regard to instep strap 12 of brace 8 (FIG. 2), this is shown separated from its clip 18. In use, end 19 of strap 12 is inserted into clip 18 to give the desired length of strap across the instep of the user. The strap can be further tensioned by the over center lever arrangement 20.

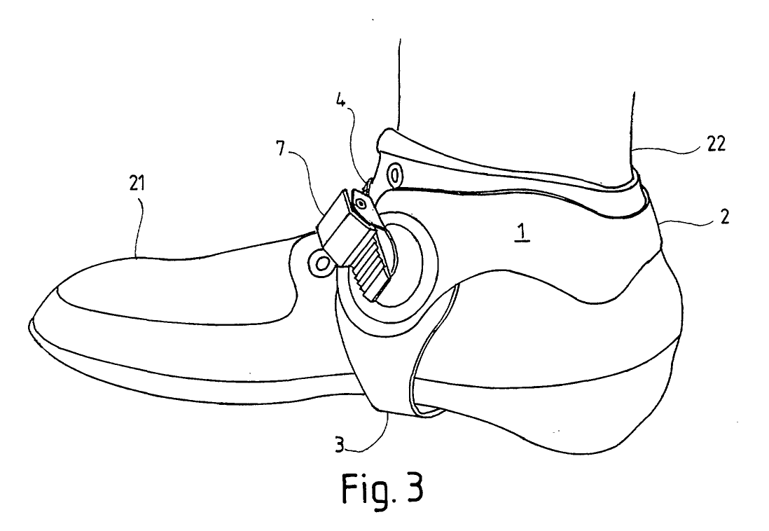

Brace 1 in position about a shoe 21 of a user is presented in FIG. 3 in which it can be seen that there is bracing of the upper portion of the shoe about the ankle 22 of the user by virtue of triangulation of forces applied by heel strap 2, sole strap 3 and instep strap 4. The adjustability of straps 3 and 4, and the tensionability of instep strap 4, gives very effective bracing of the foot. The brace can be easily removed and fitted to shoe 21 by virtue of instep strap 4 being releasable from clip 7. Furthermore, the separability of the portions (see FIG. 1) of sole strap 3 would permit brace 1 to be fitted to shoe 21 if there were a skate blade fitted to the sole of the shoe.

It will be appreciated that many changes can be made to the brace and use thereof as exemplified above without departing from the broad ambit and scope of the invention.

The term “comprise” and variants thereof such as “comprising” and “comprised” are used herein to denote the inclusion of a stated integer or integers, unless in the context of usage an exclusive interpretation of a term is required.

1. A footwear brace comprising a heel strap, a length adjustable sole strap, and a length adjustable and tensionable instep strap, wherein one end of each of said heel, sole and instep straps are joined at a common point and the other end of each of said heel, sole and instep straps are joined at another common point.

2. The brace according to claim 1, wherein said length adjustable straps each comprise two portions which portions can be releasably fixed to each other.

3. The brace according to claim 2, wherein said releasable fixing comprises a plurality of pins on one strap portion which are received in complementary holes in said other strap portion.

4. The brace according to claim 1, wherein a buckle or clasp is provided to allow length adjustment of a said strap.

5. The brace according to claim 1, wherein said instep strap has a serrated upper surface, which is acted on by a clip comprising an over-centre lever.

6. The brace according to claim 1, wherein ends of said heel strap and sole strap are pivotally connected.

7. A footwear brace comprising:
a heel strap;
a sole strap, the ends of which are pivotally connected to opposite ends of said heel strap, said sole strap comprising overlapping portions which can be releasably fixed to each other to adjust the length of said strap; and
an instep strap, which extends from one said pivotal connection of said heel strap and said sole strap and can be releasably and adjustably received by a clip on the other said pivotal connection of said heel strap and said sole strap.

8. The footwear brace according to claim 7, wherein:
said releasable fixing of said sole strap portions comprises a plurality of pins on one strap portion which are received in complementary holes in said other strap portion; and
said instep strap has a serrated outer surface which is engaged by an over-centre lever comprising said clip.

9. A pair of footwear braces comprising left and right braces according to claim 1.

10. A pair of footwear braces comprising left and right braces according to claim 7 or claim 8.
